SEQUENCE LISTING (21 sequences)

SEQ ID NO: 1 - protein, 10 aa, Unknown
  Mammallian
  VARIANT (1)..(1): Xaa = pGlu (pyroglutamic acid or pyrrolidone carboxylic acid)
XHWSYGLRPG

SEQ ID NO: 2 - protein, 10 aa, Unknown
  Mammalian
  VARIANT (1)..(1): Xaa = pGlu (pyroglutamic acid or pyrrolidone carboxylic acid)
XHWSYWLRPG

SEQ ID NO: 3 - protein, 10 aa, Unknown
  Mammalian
  VARIANT (1)..(1): Xaa = pGlu (pyroglutamic acid or pyrrolidone carboxylic acid)
XHWSYELRPG

SEQ ID NO: 4 - protein, 10 aa, Unknown
  Mammalian
  VARIANT (1)..(1): Xaa = pGlu (pyroglutamic acid or pyrrolidone carboxylic acid)
XHWSYKLRPG

SEQ ID NO: 5 - protein, 10 aa, Unknown
  Mammalian
EHWSYGLRPG

SEQ ID NO: 6 - protein, 10 aa, Unknown
  Mammalian
HHWSYGLRPG

SEQ ID NO: 7 - protein, 10 aa, Unknown
  Mammalian
PHWSYGLRPG

SEQ ID NO: 8 - protein, 5 aa, Unknown
  Mammalian
  VARIANT (1)..(1): Xaa = pGlu (pyroglutamic acid or pyrrolidone carboxylic acid)
XHWSY

SEQ ID NO: 9 - protein, 6 aa, Unknown
  Mammalian
  VARIANT (1)..(1): Xaa = pGlu (pyroglutamic acid or pyrrolidone carboxylic acid)
XHWSYG

SEQ ID NO: 10 - protein, 7 aa, Unknown
  Mammalian
  VARIANT (1)..(1): Xaa = pGlu (pyroglutamic acid or pyrrolidone carboxylic acid)
XHWSYGL

SEQ ID NO: 11 - protein, 7 aa, Unknown
  Mammalian
HWSYGLR

SEQ ID NO: 12 - protein, 6 aa, Unknown
  Mammalian
WSYGLR

SEQ ID NO: 13 - protein, 7 aa, Unknown
  Mammalian
SYGLRPG

SEQ ID NO: 14 - protein, 6 aa, Unknown
  Mammalian
YGLRPG

SEQ ID NO: 15 - protein, 10 aa, Gallus gallus
  VARIANT (1)..(1): Xaa = pGlu (pyroglutamic acid or pyrrolidone carboxylic acid)
XHWSHGWYPG

SEQ ID NO: 16 - protein, 10 aa, Unknown
  Salmo sp.
  VARIANT (1)..(1): Xaa = pGlu (pyroglutamic acid or pyrrolidone carboxylic acid)
XHWSYGWLPG

SEQ ID NO: 17 - protein, 9 aa, Unknown
  Mammalian
KWSYGLRPG

SEQ ID NO: 18 - protein, 10 aa, Unknown
  Mammalian
CKWSYGLRPG

SEQ ID NO: 19 - protein, 20 aa, Unknown
  Mammalian
GPRLGYSWKCCKWSYGLRPG

SEQ ID NO: 20 - protein, 20 aa, Unknown
  Mammalian
  VARIANT (1)..(1): Xaa = pGlu (pyroglutamic acid or pyrrolidone carboxylic acid) or Gln
XHWSYGLRPGQHWSYGLRPG

SEQ ID NO: 21 - protein, 21 aa, Unknown
  Mammalian
  VARIANT (1)..(1): Xaa = pGlu (pyroglutamic acid or pyrrolidone carboxylic acid)
  VARIANT2 (6)..(6): Xaa = Gly or D-amino acid which may optionally contain an amino acid side chain
  VARIANT3 (16)..(16): Xaa = Gly or D-amino acid which may optionally contain an amino acid side chain
XHWSYXLRPGQHWSYXLRPGC

(19) United States
(12) Patent Application Publication
(10) Pub. No.: US 2022/0016225 A1
(43) Pub. Date: Jan. 20, 2022

(54) VACCINE COMPOSITIONS AND ADJUVANT

(71) Applicant: The United States as Represented by the Secretary of Agriculture, Washington, DC (US)

(72) Inventors: Lowell A. Miller, Greeley, CO (US); Jack C. Rhyan, Fort Collins, CO (US)

(21) Appl. No.: 16/933,157

(22) Filed: Jul. 20, 2020

(51) Int. Cl. A61K 39/00; C07K 14/59; A61K 47/62

(52) U.S. Cl. CPC A61K 39/0006; C07K 14/59; A61K 47/62; A61K 2039/552; A61K 2039/55505; A61K 2039/54; A61K 2039/521; A61K 2039/55594

(57) ABSTRACT

The immune response of an animal to a target immunogen may be enhanced by use of an adjuvant which includes low concentrations of killed cells of Mycobacterium avium subspecies avium in combination with mineral oil. The adjuvant may be used in vaccine compositions for the immunization of an animal against any target immunogen and is particularly preferred for use with immunocontraceptive vaccines such as GnRH immunocontraceptive vaccines conjugated with a Blue carrier protein.

### BACKGROUND

### 1. Field

The invention relates to novel vaccine compositions, including immunocontraceptive vaccines, and particularly to novel adjuvants for use therein.

### 2. Description of the Related Art

Gonadotropin releasing hormone (“GnRH”, also known as Luteinizing Hormone Releasing Hormone, or “LHRH”), has long been recognized as being of central importance to the regulation of fertility in animals. GnRH is a decapeptide which has the same amino acid sequence, i.e., pGlu-His-Trp-Ser-Tyr-Gly-Leu-Arg-Pro-GlyNH₂ (SEQ ID NO. 1) in all mammals. Closely related GnRH compounds have also been identified in other non-mammals, including fowl, and receptors for GnRH have been identified in reptiles and amphibians. In males and females, GnRH is released from the hypothalamus into the bloodstream and travels via the blood to the pituitary, where it induces the release of the gonadotropins, luteinizing hormone (LH) and follicle stimulating hormone (FSH). These two gonadotropins, in turn, act upon the gonads, inducing steroidogenesis and gametogenesis. In growing male animals, the gonadotropins stimulate the development of the testes and the synthesis of testicular steroids. In the growing female animal the development of the ovaries is stimulated, providing therein follicle development, synthesis of ovarian steroids and ovulation. Steroids released from the gonads into the circulation also act upon various other tissues.

In recent years, GnRH neutralization has been used as an effective means of contraception in a variety of animals. Fraser described that the gonadotropin hormonal cascade can be halted by neutralization of the biological activity of GnRH (Physiological Effects of Antibody to Luteinizing Hormone Releasing Hormone. In: Physiological Effects of Immunity Against Reproductive Hormones, Edwards and Johnson, Eds. Cambridge University Press, 1976). As a consequence of GnRH neutralization, the gonadotropins and gonadal steroids are not released into the blood, interrupting the hormonal regulation of fertility and ceasing gametogenesis. In addition to the use of immunization against GnRH for animal sterilization to prevent breeding, the immunization has also been suggested for the treatment of aggressiveness in male animals such as dogs and bulls, chemical castration of male animals for slaughter, prevention of heat in female animals, prevention of restlessness in male animals being fattened for slaughter, and reduction of boar taint in the meat of pigs raised for slaughter.

Neutralization of GnRH has also been employed for the treatment of a number of other diseases. A number of important diseases, including breast cancer, uterine and other gynecological cancers, endometriosis, uterine fibroids, prostate cancer, and benign prostatic hypertrophy, are also affected by gonadotropins and gonadal steroid hormones. Neutralization of the patient's GnRH effectively eliminates the gonadal steroids that induce and stimulate these diseases. See McLachlan et al., 1986, British Journal of Obstetrics and Gynecology, 93:431-454; Conn and Crowley, 1991, New England Journal of Medicine, 324:93-103; Filicori and Flamigni, 1988, Drugs, 35:63-82.

GnRH neutralization has been typically achieved by the induction or introduction of anti-GnRH antibodies in the subject animal or patient. These antibodies may be induced by active immunization with GnRH immunogens, or by passive immunization by administering anti-GnRH antibodies (Fraser, 1976, ibid). Antibodies to GnRH produce infertility by binding to circulating endogenous GnRH, precluding the GnRH from binding to its pituitary receptor and thereby interfering with its ability to release FSH and LH. The severe reduction or absence of these hormones leads to atrophy of the gonads and concomitant infertility in both sexes as described above.

Despite these advantages, active immunization against GnRH has not been widely practiced due to deficiencies associated with the GnRH vaccines. The prior art anti-GnRH vaccines have typically lacked the potency necessary to effect long-term contraception in a single dose. In fact, immunocontraception has traditionally required at least two doses, a prime and a boost, for long-term efficacy. The prime dose prepares the immune system for repeat antigen exposure and provides only a short term response. The subsequent boost immunization can result in an immune response which can be maintained for a period of months to years. In addition to the GnRH immunogen, an adjuvant is a necessary component of any vaccine intended for long-lasting immunocontraception.

At present, Freund's complete adjuvant (FCA) is the only adjuvant that has provided high and long-lasting immunocontraceptive responses. Although many other adjuvants have been developed, none have been able to achieve the high antibody titers obtained using Freund's complete adjuvant. Freund's complete adjuvant includes an emulsion of killed bacteria of Mycobacterium tuberculosis or M. butyricum (also known as M. smegmatis) in mineral oil with a surfactant.

Despite the efficacy achieved with Freund's complete adjuvant, numerous concerns have been raised over its use in animals, and particularly in animals raised for human consumption. One primary concern has been the potential for false-positive TB skin tests in an animal which has been injected with FCA containing killed M. tuberculosis (Tizard, 1977, An Introduction to Veterinary Immunology, CRC Press, Boca Raton, Fla.). Other concerns over the use of FCA have included fears that it may be carcinogenic and that it may cause intense cell-mediated immune responses which produce lesions at the site of injection.

### SUMMARY

The present subject matter relates to methods and compositions for vaccinating animals. In accordance with this subject matter, the immune response of an animal to a target immunogen may be enhanced by use of an adjuvant which includes low concentrations of killed cells of Mycobacterium avium subspecies avium or Mycobacterium avium complex in combination with mineral oil. While the adjuvant may be used in vaccine compositions for the immunization of an animal against any target immunogen, it is particularly preferred for use with immunocontraceptive vaccines targeting immunogens such as gonadotropin releasing hormone (GnRH). The vaccine may include an immunogen, such as by way of non-limiting example GnRH or a GnRH immogenic analog, which may be conjugated to a carrier protein, for example, a Blue carrier protein. In other embodiments, the immunogen may be conjugated to a KLH-carrier protein.

In another embodiment, it is an object of the present subject matter to provide vaccine compositions having this adjuvant and conjugated vaccine.

Another object of the present subject matter is to provide an adjuvant for use with conjugated vaccine compositions which provides superior enhancement of immune response to the target immunogen but which produces substantially no inflammation at the site of injection. A further object is to provide conjugated vaccine compositions having an adjuvant in an amount effective for inducing immunocontraception in a high percentage of vaccinated animals using a single dose, without a second or boost dose, for a period of at least one year.

Still another object is to provide a vaccine composition for the immunocontraception of animals.

Yet another object is to provide novel contraceptive vaccine compositions which are effective for long periods of time with only a single shot.

Other objects and advantages of the present subject matter will become readily apparent from the ensuing description.

### BRIEF DESCRIPTION OF THE DRAWINGS

FIG. 1 shows the results of the first study of Example 5 over four consecutive seasons with deer immunized with a prime dose followed by a second, boost dose.

FIG. 2 shows the results of the second study of Example 5 over two seasons with female deer using only a single shot of the vaccine.

FIG. 3 shows the results of the study of Example 6 with female pigs.

FIGS. 4(a) and 4(b) shows the results of the study of Example 6 with female pigs.

### DETAILED DESCRIPTION

In accordance with the present subject matter we have developed novel adjuvants for use in the active immunization of animals. Traditionally, vaccines are prepared using a combination of an immunologically effective amount of an immunogen of interest together with an adjuvant effective for enhancing the immune response of the animal against the immunogen. We have unexpectedly discovered that compositions of mineral oil with low concentrations of killed cells of Mycobacterium avium subspecies avium or Mycobacterium avium complex provide effective enhancement of immune responses and thus are effective for use as adjuvants. Moreover, this adjuvant provides enhanced immune responses which exhibit high, long-lasting antibody titers to the immunogen, even after administration of only a single dose of the vaccine. Therefore, the concentration of the killed cells of Mycobacterium avium are present in an amount effective for inducing immunocontraception of an animal in a single dose.

As used herein Mycobacterium avium subspecies avium refers to the recognized species M. avium subspecies avium, the characteristics of which are described by Thorel et al. (1990, International Journal of Systematic Bacteriology, 40:254-260, the contents of which are incorporated by reference herein) and the type strain of which has been deposited at the American Type Culture Collection, Manassas, Va., USA, as deposit accession number ATCC 25291. As used herein, Mycobacterium avium complex collectively includes both of the two subspecies M. avium subsp. avium and M. avium subsp. paratuberculosis as well as M. intracellulare.

It is believed that the high efficacy of the M. avium subsp. avium or Mycobacterium avium complex containing adjuvant is due to the nearly ubiquitous presence of this microorganism in nature. Indeed, most living animals, including humans, have been exposed to M. avium subsp. avium in the environment. We believe that because most animals have been naturally exposed to the microorganism, when they are initially injected with the immunogen plus adjuvant the immune response is enhanced by a specific response to the M. avium subsp. avium or Mycobacterium avium complex which is similar to that of a booster injection. The initial injection with the present adjuvant therefore elicits an immune response which is usually seen only after a boost injection of other adjuvants.

The particular strain of M. avium subsp. avium used in the preparation of the adjuvant is not critical. M. avium subsp. avium suitable for use in the adjuvant may be obtained from a variety of sources including known substantially pure strains or it may be isolated from natural sources such as fowl or other animals using conventional techniques. For commercial production of the adjuvant, large quantities of cells of the microorganism are preferably prepared by culture of the selected strain. Alternatively, killed cells may be obtained directly from commercial sources, such as the Johne's disease vaccine, MYCOPAR (originally manufactured by Fort Dodge Animal Health, Overland Park, Kans., USA, now Boehringer Ingelheim, Ridgefield, Conn., USA), which consists of killed cells M. avium subsp. avium strain 18.

Propagation of the microorganism for preparation of the adjuvant may be accomplished by culture under any conventional conditions and on media which promote its growth. Although a variety of conventional solid and liquid media may be suitable for use herein, growth in liquid culture is particularly preferred for large scale production. Without being limited thereto, Middlebrook broth is preferred. The microorganism will grow over wide temperature ranges, although growth between 34-38° C. is typically preferred. Once a sufficiently heavy growth of the microorganism has been obtained, usually in about 10-25 days, the cells may be recovered and separated from the culture medium using techniques conventional in the art, such as by centrifugation or filtration. Following separation, the cells may be further washed to remove contamination by extraneous microbial products or culture media components.

Following their propagation and recovery, cells of M. avium subsp. avium or M. avium complex are subjected to chemical and/or physical treatment effective to kill (i.e., inactivate) the cells. An effective treatment for killing the cells is defined herein as that which kills 99.9% or more of the viable cells, without lysing the cells and while retaining the ability of the cells to elicit an antibody response in the animal. Thus, the treatment should not substantially alter the specificity of the cell surface antigens on the killed cells relative to the untreated cells. While treatments killing 100% of all viable cells would typically be preferred, particularly for any applications involving treatment of humans, the skilled practitioner will recognize 100% cell death may not be critical in veterinary applications, particularly in view of the ubiquitous nature of the microorganism.

In an embodiment, killed, intact M. avium subsp. avium are prepared by treatment of the viable cells with alcohol, particularly an aliphatic alcohol such as ethanol or isopropyl alcohol. In another embodiment, killed, intact M. avium complex are prepared by treatment of the viable cells with alcohol, particularly an aliphatic alcohol such as ethanol or isopropyl alcohol. Alternatively, the cells may be killed by UV irradiation such as described by Purdy et al. (U.S. Pat. No. 6,303,130) for the preparation of Pasteurella haemolytica bacterins. It also envisioned a variety of other techniques that have been described for the preparation of killed cell vaccines (i.e., bacterins) are also suitable for use herein, and include but are not limited to treatment with phenol, tricresol, formalin, formaldehyde, acetone, merthiolate, and moderate heat at temperatures which would not induce protein denaturation (e.g., 56° C. for 1 hour). Treatment times and conditions will of course vary with the particular method selected and may be readily determined by routine testing.

Adjuvant formulations are prepared by combining the killed cells with mineral oil, which is also used in the preparation of the well-known Freund's adjuvant, and an optional surfactant or emulsifier. Inclusion of a surfactant is indicated when either or both of the M. avium subsp. avium or Mycobacterium avium complex killed cells or the immunogen of interest are in an aqueous solution or suspension. A variety of surfactants are suitable for use herein for emulsifying any aqueous components. Although mannide monooleate is a generally preferred surfactant, examples of alternative surfactants which may also be used include but are not limited to isomannide monooleate, aluminum monostearate, polyoxyethylene ethers (or octoxynols) such as lauryl, cetyl, oleyl, stearyl, and tridecyl polyoxyethylene ethers; polyoxyethylene sorbitan-fatty acid esters (commonly sold under the trade name TWEEN by ICI Americas Incorporated, Wilmington, Del.), such as polyoxyethylene(20)sorbitan monolaurate (TWEEN 20), polyoxyethylene(60)sorbitan monolaurate (TWEEN 60); polyoxyethylene ethers such as TRITON X-100, X-102, X-165, and X-305; fatty acid diethanolamides such as isostearic acid DEA, lauric acid DEA, capric acid DEA, linoleic acid DEA, myristic acid DEA, oleic acid DEA, and stearic acid DEA; fatty acid monoethanolamides such as coconut fatty acid monoethanolamide; fatty acid monisopropanolamides such as oleic acid monoisopropanolamide and lauric acid monoisopropanolamide; alkyl amine oxides such as N-cocodimethylamine oxide, N-lauryl dimethylamine oxide, N-myristyl dimethylamine oxide, and N-stearyl dimethylamine oxide; N-acyl amine oxides such as N-cocoamidopropyl dimethylamine oxide and N-tallowamidopropyl dimethylamine oxide; N-alkoxyalkyl amine oxides such as bis(2-hydroxyethyl) C₁₂-C₁₅ alkoxy-propylamine oxide, and combinations thereof. In some embodiments, the surfactant is selected from the group consisting of mannide monooleate, isomannide monooleate, aluminum monostearate, and combinations thereof. The amount of the surfactant, if used, is not critical but should be sufficient to emulsify any aqueous components. Consequently, the relative amounts of mineral oil to surfactant in the adjuvant will typically be between about 85:15 and about 100:0, by weight, respectively. In one embodiment, the ratio of mineral oil to surfactant may vary between about 85:15 to 95:5, respectively, or about 95:5.

In contrast to the mineral oil and surfactant, the amount of killed cells of M. avium subsp. avium or M. avium complex is critical. The objective of the adjuvant coincides with the well-established use of adjuvants in the active immunization art, which is to enhance the immune response of an animal to immunization with a target immunogen, specifically, to enhance the production of antibodies against the target immunogen. Thus, the adjuvant is administered in a vaccine composition which includes a target immunogen of interest, wherein the target immunogen is itself present in an immunologically effective amount. As used throughout the art and herein, an immunologically effective amount of the target immunogen is defined as that amount which will elicit production of antibody by the subject animal against the immunogen.

Consequently, in accordance with the present subject matter, the absolute amount of the killed cells of M. avium subsp. avium or M. avium complex and their concentration in the final vaccine composition (which includes the target immunogen) which is administered to the subject animal are selected to provide an effective enhancement (i.e., increase) of the production of the antibody against the target immunogen as compared to a control animal (treated with a vaccine composition lacking the adjuvant). However, the amount of the killed cells of M. avium subsp. avium or M. avium complex in the vaccine composition should not be so high that it would elicit a substantial T cell-mediated delayed hypersensitivity response (i.e., a Type IV response) by the animal to the M. avium if the adjuvant were administered alone without immunogen.

A substantial T cell-mediated delayed hypersensitivity response to M. avium subsp. avium or M. avium complex is defined herein as a skin reaction at the site of injection of the vaccine which is visible to the naked eye. Although the administration of the present adjuvant may produce a reaction on a microscopic level which may be seen upon microscopic examination of biopsy material, in some species no granuloma at the site of injection will be visible to the naked eye. Thus, the amount of the killed cells used in the vaccine will be much less than that which might be typically used for a target immunogen. The precise effective amount of the killed cells used in the vaccine composition may vary somewhat with the particular target animal and its size, and the stage of the vaccination (initial or single dose, or second or boost dose) and may be determined by the practitioner in the art by routine experimentation. In all instances, though, the amount of the killed cells used in the vaccine composition is effective for inducing immunocontraception of an animal in a single dose.

Without being limited thereto, the concentration of said killed cells of M. avium in the vaccine formulation administered to a subject animal will typically vary between about 50 μg per ml and about 400 μg per ml, measured as the dry weight of said killed cells per ml of the vaccine composition. In the alternative, the concentration of said killed cells of M. avium in the vaccine formulation administered to a subject animal will typically be less than or equal to about 400 μg per ml, measured as the dry weight of said killed cells per ml of the vaccine composition. Within this range, the initial dose of vaccine formulations administered to an animal in either a single or multiple dose program will generally have a greater amount of killed cells of M. avium, than boost doses. Further, as a practical matter, the volume of vaccine compositions which may be administered to animals parenterally by injection is relatively small, no greater than about 1 ml for all but very large animals such as horses, elephants or whales. Consequently, the volume of adjuvant and hence the amount of killed cells of M. avium in the vaccine composition is also limited. Thus, in one embodiment for the treatment of animals weighing less than about 2,000 pounds or, in certain embodiments, weighing less than about 1,000 pounds, (i.e., animals except the above-mentioned very large animals), the vaccine composition will typically contain more than or equal to about 50 μg and less than or equal to about 400 μg of killed cells of M. avium by weight, and particularly more than or equal to about 50 μg and less than or equal to about 200 μg of the killed cells by weight, measured as the dry weight of said killed cells.

The manner of formulating the adjuvant and immunogen containing preparation may vary with the phase of the immunogen preparation, the concentration of the immunogen in the preparation, its solubility, and the carrier used for the immunogen preparation, if present. For instance, without being limited thereto, when formulated with aqueous phase solutions or suspensions of an immunogen, the adjuvant and immunogen preparation are preferably formulated in approximately equal volumes, vigorously agitated to form an emulsion, and finally stiffened by passage through a needle as is conventional in the art. Immunogen preparations in oil miscible carriers may also be simply mixed with adjuvant, again preferably in approximately equal volumes.

The present M. avium subsp. avium or M. avium complex containing adjuvants may be used in combination with a variety of known immunogens or antigens used for active immunization of animals by parenteral injection to elicit production of antibodies reactive with the immunogen. In one embodiment, the adjuvants can be used with GnRH, and particularly with GnRH or porcine zona pellucida (PZP) immunocontraceptive vaccines. However, it is also envisioned that the adjuvants may be used with virtually any other known immunogen of interest other than M. avium (such as Johne's disease vaccine). Thus, the adjuvant may be used with pathogenic microorganisms (living, attenuated, or killed) or biological molecules including toxoids, polysaccharides, proteins, peptides, or microbial subunits, which further include relatively large molecules which are themselves antigenic in a target animal as well as smaller haptens or self-molecules conjugated to immunogenic carriers. Examples of immunogens used in vaccination programs which may be used with the present adjuvants include but are not limited to Pasteurella haemolytica, Vibrio cholera, Corynebacterium diptheriae toxoid, Hepatitis B viral antigen, Influenza virus, Measles virus, Meningococcal polysaccharide, Mumps virus, killed cells of Bordetella pertusis, Streptococcus pneumoniae (pneumococcus) polysaccharide, Polio viruses, Rabies virus, Rubella virus, poxviruses such as Vaccinia virus, Clostridium tetani toxoid, Mycobacterium bovis, killed cells of Salmonella typhi, and Yellow fever virus.

In one embodiment, the adjuvant is formulated with a GnRH or GnRH immunogenic mimic containing vaccine for inducing production of anti-GnRH antibody in an animal and thereby effecting one or more responses ranging from contraception in males and/or females, reducing aggressive behavior in male animals, chemical castration, control or prevention of estrus or heat, prevention of restlessness in animals prior to slaughter, reduction of boar taint in the meat of pigs raised for slaughter, and treatment of diseases as is known in the art.

While GnRH may be used in the immunogen preparation, a variety of GnRH immunogenic analogs have also been described which are suitable for use herein. As noted hereinabove, GnRH is a small decapeptide having the amino acid sequence: pGlu-His-Trp-Ser-Tyr-Gly-Leu-Arg-Pro-Gly-NH₂ (Seq. ID No. 1). Throughout this description, the amino acid sequences conform with conventional practice with the amino terminal amino acid on the left and the carboxy terminal amino acid to the right. As defined herein, immunogenic analogs of GnRH include compounds containing a substitution, deletion, or insertion of between one and five amino acid residues in the above-mentioned GnRH amino acid sequence, as well as dimers or polymers thereof, which compound retains the ability to induce or stimulate the production in a subject animal of antibodies which bind (i.e., cross-react) to GnRH. The GnRH analog will preferably retain at least five consecutive amino acids from the GnRH decapeptide. The substitutions and insertions can be accomplished with natural or non-natural amino acids, and substitutions are preferably conservative substitutions made with amino acids which maintain substantially the same charge and hydrophobicity as the original amino acid. Moreover, the analog may itself be immunogenic or it may be coupled to an immunogenic carrier such as described hereinbelow.

Immunogenic analogs of GnRH which are suitable for use herein have been described, for example, in Meleon (U.S. Pat. Nos. 5,484,592 and 6,284,733), Mia (U.S. Pat. No. 4,608,251), Ladd et al. (U.S. Pat. No. 5,759,551), Hoskinson et al. (published PCT application WO8805308), and Russell-Jones et al. (U.S. Pat. No. 5,403,586) the contents of each of which are incorporated by reference herein. Thus, suitable GnRH analogs include but are not limited to GnRH peptides wherein the Gly at position 6 of the GNRH decapeptide has been replaced by a dextrorotary (D)-amino acid such as D-trp, D-glu, or D-lys (Seq. ID No. 2, 3, and 4, respectively); GnRH peptides wherein the p-Glu at position 1 of the GnRH decapeptide has been replaced by a Glu, His, or Pro (Seq. ID No. 5, 6, and 7, respectively); any continuous 5, 6, 7, 8, or 9 amino acid fragment of the GnRH decapeptide, such as pGlu-His-Trp-Ser-Tyr, pGlu-His-Trp-Ser-Tyr-Gly, pGlu-His-Trp-Ser-Tyr-Gly-Leu, His-Trp-Ser-Tyr-Gly-Leu-Arg, Trp-Ser-Tyr-Gly-Leu-Arg, Ser-Tyr-Gly-Leu-Arg-Pro-Gly-NH₂, and Tyr-Gly-Leu-Arg-Pro-Gly-NH₂ (Seq. ID Nos. 9-15, respectively); naturally occurring chicken GnRH II, pGlu-His-Trp-Ser-His-Gly-Trp-Tyr-Pro-Gly-NH₂ (Seq. ID No. 16); naturally occurring salmon GnRH, pGlu-His-Trp-Ser-Tyr-Gly-Trp-Leu-Pro-Gly-NH₂ (Seq. ID No. 17); the nona- or decapeptide (Cys)-Lys-Trp-Ser-Tyr-Gly-Leu-Arg-Pro-Gly-NH₂, wherein the amino terminal Cys is optional (Seq. ID Nos. 18 and 19, respectively) or a dimer of the decapeptide wherein the amino terminal Cys are coupled to one another (Seq. ID No. 20); a polymer of two or more decapeptides in tandem of the formula Z¹-Glx-His-Trp¹-Ser-Tyr-Gly-Leu-Arg-Pro[-Gly-X-Gln-His-Trp-Ser-Tyr-Gly-Leu-Arg-Pro]ₙ-Gly-Z² wherein n is an integer greater than or equal to 1, X is a direct bond or a spacer, Z¹-Glx is pGlu or Gln having an amino acid tail attached thereto for coupling to a carrier protein, and Gly-Z² is Gly-NH₂ or Gly having an amino acid tail attached thereto for coupling to a carrier protein (Seq. ID No. 21); and a peptide having the sequence pGlu-His-Trp-Ser-Tyr-Y-Leu-Arg-Pro-Gly-Gln-His-Trp-Ser-Tyr-Y-Leu-Arg-Pro-Gly-Cys wherein Y is independently Gly or a D-amino acid which may optionally contain an amino acid side chain attached thereto for coupling to a carrier protein (Seq. ID No. 22) or a dimer thereof.

While the GnRH may be isolated from natural sources, for practical purposes GnRH or and its analogs may be synthesized by a variety of conventional methods. Such techniques include but are not limited to methods well known to those skilled in the art of peptide synthesis, e.g., solution phase synthesis [see Finn and Hoffman, In “Proteins,” Vol. 2, 3rd Ed., H. Neurath and R. L. Hill (eds.), Academic Press, New York, pp. 105-253 (1976)], or solid phase synthesis [see Barany and Merrifield, In “The Peptides,” Vol. 2, E. Gross and J. Meienhofer (eds.), Academic Press, New York, pp. 3-284 (1979)], or stepwise solid phase synthesis as reported by Merrifield [J. Am. Chem. Soc. 85: 2149-2154 (1963)], the contents of each of which are incorporated herein by reference.

Because GnRH is a small, self-molecule it should be conjugated directly or indirectly to an immunogenic carrier in order to increase the immune response to the peptide. A plurality of carriers and carrier coupling techniques have been previously described for GnRH or its analogs and are also suitable for use herein. See for example, Meleon, Mia, Ladd et al., Hoskinson et al., and Russell-Jones et al. mentioned above. However, in one embodiment, GnRH or an analog thereof is conjugated to immunogenic mollusk hemocyanin carrier protein, directly or indirectly through the C-terminal end of the GnRH or analog. Suitable immunogenic mollusk hemocyanin proteins include Concholepas concholepas hemocyanin protein, Keyhole Limpet (Megathura crenulate) hemocyanin protein (KLH), Concholepas concholepas Hemocyanin (Blue), Horseshoe crab (Limulus polyphemus) hemocyanin protein, and Abalone (Haliotis tuberculata) hemocyanin protein. In an embodiment, the hemocyanin protein is Concholepas concholepas Hemocyanin (Blue), also described a “Blue protein” or “Blue carrier protein” herein. Blue protein may be obtained in activated and non-activated forms through BIOSONDA Biotechnology®.

The Blue protein exhibits most of the same immunogenic properties as KLH, but provides better solubility resulting in more flexibility by allowing a broader range of buffer and pH conditions for conjugation. Specifically, Blue protein is the high molecular mass respiratory glycoprotein obtained from the hemolymph of the marine mollusk Concholepas concholepas. The large size of the Blue protein, which has a native didecamer mass of approximately 8,000 kD, provides for efficient endocytosis by antigen presenting cells (APCs) wherein it is processed into peptides, bound to major histocompatibility complex (MHC) class II molecules, and presented to the immune system on the APC surface membrane. APC presentation of antigenic peptides in the context of MHC class II initiates the binding, priming and proliferation of CD4+T helper cells, driving T cell lymphokine secretion and a cascade of cellular and humoral responses. Unlike many other carrier proteins, Blue protein is made by two different polypeptides CCA-A and CCH-B, thus conferring to the molecule more stability which makes possible the generation of a broader variety of immunogenic peptides after processing by APCs.

Conjugation of GnRH or its analog to the mollusk hemocyanin protein is preferably conducted using a cross-linking agent to allow a large number of GnRH or analog molecules (i.e., 200 or more) to be coupled to a single carrier protein molecule, effectively covering its outer surface with consistently aligned epitopes of the GnRH displaying the same basic conformation. To ensure this consistent alignment, the GnRH (or its analog) is coupled through its C-terminal end to the N-terminal end of the carrier protein through a bifunctional cross-linking agent. In one embodiment, the GnRH/carrier conjugate may be shown by the formula:

(X-Aₘ-B-L)ₙ-R (I)

wherein X is GnRH or a GnRH immunogenic analog, A is an optional amino acid spacer such as Gly, m is an integer greater than or equal to 0, B is an amino mercaptan, R is an intact immunogenic mollusk hemocyanin protein, L is a bifunctional crosslinking agent effective for simultaneously binding to the thiol of the mercaptan and to free amine moieties of the immunogenic mollusk hemocyanin protein, and n is an integer greater than or equal to about 200. A variety of amino mercaptans may be used, provided that it possesses a free amino moiety for binding to the C-terminal end of X (or A if present) and a free thiol moiety for binding to the bifunctional crosslinking agent. In one embodiment, the amino mercaptan is cysteine. Nonlimiting of suitable examples, bifunctional crosslinking agents include, without limitation, succinimidyl-4-(N-maleimidomethyl)cyclohexane-1-carboxylate (SMCC) or sulfo-SMCC (s-SMCC), either of which form a maleimide-activated carrier protein. Other crosslinking agents suitable for conjugating the carrier protein and GnRH through the thiol group of the amino mercaptan include but are not limited to the organic solvent soluble agents Succinimidyl 4-(p-maleimidophenyl)-butyrate (SMPB), -[(γ-Maleimidobutyryl)oxy]succinimide ester (GMBS), -Succinimidyl[4-iodoacetyl]-aminobenzoate (SIAB), and m-Maleimidobenzyl-N-hydroxysuccinimide ester (MBS), or their corresponding water soluble sulfonated forms sulfo-SMPB (s-SMPB), sulfo-GMBS (sGMBS), sulfo-SIAB (s-SIAB), and sulfo-MBS (s-MBS).

Preparation of the above-mentioned GnRH/mollusk hemocyanin protein conjugate is preferably conducted under conditions of approximately neutral pH and high salt concentrations to prevent the disassociation of the protein into subunits, and thereby prevent mollusk protein epitopes from being exposed to the animal's immune system. Thus, the protein is preferably dissolved in a buffer having an NaCl concentration greater than or equal to about 0.6 M, particularly about 0.9 M. A detailed description of the conjugation procedure is provided in Example 2.

The GnRH or GnRH analog carrier conjugate is formulated with the present adjuvant in the same manner described above for any immunogen of interest. However, when using compositions which include a mollusk hemocyanin carrier protein, the vaccine composition may further include physiologically buffered saline with a high salt concentration to prevent dissociation of the protein. The salt (NaCl) concentration of the vaccine composition may be greater than or equal to about 0.7 M and less than or equal to about 1.0 M, and the pH of said vaccine composition may be between about 7.0 and 8.0. In an embodiment, the PH is about 7.4.

In other embodiments, when using compositions which include a mollusk hemocyanin carrier protein, the immunogen component of the vaccine composition may further include an antibiotic or preservative. This can help to maintain the stability and storage-stability of the vaccine composition.

In this embodiment, the present subject matter provides a method for inducing anti-GnRH antibody by administering to a subject animal a vaccine composition including the adjuvant containing killed cells of M. avium subsp. avium or M. avium complex with the GnRH or GnRH analog conjugate. As noted, the production of the anti-GnRH antibodies effects the neutralization of GnRH in the animal, thereby reducing LH and FSH blood levels and inhibiting the production of androgens and other steroids and sperm in the testes of males, and inhibiting the production of progestogens and oestrogens and follicle maturation in the ovary of females. As a consequence, the induction of anti-GnRH antibodies may be used for effecting one or more treatments of animals, including the contraception of males and/or females, reducing aggressive behavior in male animals, chemical castration, control or prevention of estrus or heat, prevention of restlessness in animals prior to slaughter, reduction of boar taint in the meat of pigs raised for slaughter, and treatment of various diseases as noted hereinabove.

Accordingly, the GnRH or GnRH analog conjugate should be administered in an amount effective to induce one or more of these responses as determined by routine testing. For example, where the desired effect is contraception, an “effective amount” is defined to mean those quantities which will result in a significant reduction in fertility relative to an untreated control animal. Infertility can be measured by methods known in the art, e.g. by evaluation of spermatogenesis or ovulation, as well as by statistical modeling of experimental animal data. Other indicators of infertility in males includes reduction of serum testosterone to castration levels and involution of the testes. An effective amount can also include those amounts which do not result in complete infertility, but which render an animal unable to carry a pregnancy to a full term. Similarly, where the ultimate response is a reduction of aggressive behavior in male animals, an effective amount is defined to mean those quantities which will result in a significant reduction in aggressive behavior of a test group as compared to an untreated group. The actual effective amount will of course vary with the specific GnRH or GnRH analog, the immunogenic carrier and manner of conjugation, the target animal and its size, the desired effect, and the treatment regimen (i.e., treatment with only a single dose, or treatment with a first dose followed by a boost dose), and may be readily determined empirically by the practitioner skilled in the art using an antigen dose response assay for each animal species.

Without being limited thereto, typical single shot doses of the GnRH or GnRH analog conjugate in the vaccine for the treatment of small animals (such as rodents, Norway rats, squirrels, rabbits, dogs, and domestic cats) will be between about 50 and 250 μg, for medium size animals (such as pigs or deer) will be between about 400 and 800 μg, and for large animals (such as cattle, bison, horses, or elk) will be between about 1,000 to 2,000 μg conjugate. The doses presented above are provided only as a guide for adult or full-size animals, and for animals encompassing a number of species or breeds, such as dogs, deer, or horses, the indicated dose is for the average or typical size animal, not “miniature” breeds or species. It is envisioned that doses for very large animals such as elephants would be considerably greater and should be determined empirically.

We have discovered that when the vaccine is formulated with the present killed M. avium subsp. Avium or M. avium complex adjuvant, the above-described doses of GnRH or GnRH analog provide effective immunocontraception for an extended period of time, eliciting high anti-GnRH antibody titers for periods of more than 1 year in the majority of vaccinated animals, after only a single dose or shot. For treatment programs utilizing two doses, a first primary dose and a second boost dose, the doses described above for a single dose regimen should be cut approximately in half for each dose.

The GnRH or GnRH analog containing vaccines are effective for treatment of a broad spectrum of both wild and domesticated animals, ranging from pets, to large domestic or wild animals, including mammals, birds, and reptiles. Without being limited thereto, preferred animals which may be treated include porcine, bovine, equine, feline, canine, primates (including humans), Rodentia, Cervidae, and Pachydermata, and particularly domestic dogs, domestic cats, pigs (including captive or feral pigs), cattle, deer, horses, zoo animals, elephants, rodents (including rats, rabbits, and squirrels), and reptiles.

The vaccine may be administered to the subject animal by parenteral injection (e.g., subcutaneous, intravenous, or intramuscular). For immunocontraceptive treatments, the vaccine should be administered prior to the desired onset of infertility to allow development of effective levels of anti-GnRH antibodies in the subject animal. Thus, the vaccine will typically be injected at least about 3 months prior to the desired time of contraception.

The following examples are intended only to further illustrate the present subject matter and are not intended to limit the scope of the present subject matter which is defined by the claims.

### Example 1

### Preparation of Adjuvant

Killed cells of Mycobacterium avium subsp. avium were obtained from the commercial Johne's disease vaccine, a Mycobacterium avium bacterin (previously provided by Solvay Animal Health Products Inc., Mendota Heights, Minn. 55120, Product #09149, rights now owned by Boehringer Ingelheim, Ridgefield, Conn., USA). Each vial contains a total of 25.5 mg killed cells of Mycobacterium avium (dry weight) in 0.5 ml of mineral oil. Vials are stored at refrigerator temperature, 36 to 45.degree. F.

A mineral oil diluent is used for preparation of the adjuvant. In a sterile 50 ml centrifuge tube, combine mineral oil (light white oil) and mannide monooleate (9:1 by weight) and vortex to mix. Store at room temperature in the sterile 50 ml vial.

A concentrated stock solution of the killed cells in mineral oil may be prepared for storage and subsequent preparation of adjuvant doses in vaccine formulations. The Stock Solution is a 1/30 dilution of the original Mycobacterium avium bacterin. Prepare in a sterile 50 ml centrifuge tube. Add an entire vial (0.5 ml) of Mycobacterium avium bacterin to 10 ml of the diluent. Rinse the Mycobacterium avium bacterin vial 2 times with 1 ml of diluent; each rinse is added to the 50 ml vial bringing the total volume to 12.5 ml, then add an additional 2.5 ml of diluent to the stock vial bringing the total volume to 15 ml. Vortex to mix. Vials are stored at refrigerator temperature. The concentration of killed cells in the stock solution is 1.7 mg/ml.

The stock solution is diluted as necessary to prepare both the prime and the boost dose adjuvants used in the vaccines. The primary dose adjuvant is a 1/150 dilution of original Mycobacterium avium bacterin. In a sterile 50 ml centrifuge tube, make a 1/5 dilution of the stock solution with the diluent. Add 1 ml of the Stock Solution to 4.0 ml of the diluent. Vortex to mix. The concentration of killed cells in the prime dose adjuvant is 340 μg/ml. Vials are stored at refrigerator temperature.

The boost dose Adjuvant is a 1/300 dilution of the Mycobacterium avium bacterin. In a sterile 50 ml centrifuge tube, make a 1/10 dilution of the stock solution with the diluent. Add 1 ml of the stock solution to 9.0 ml of the diluent. Vortex to mix. The concentration of killed cells in the prime dose adjuvant is 170 μg/ml. Vials are stored at refrigerator temperature.

### Example 2

### GnRH-KLH Vaccine Preparation

### Preparation of MSB Buffer:

Add 7 tablets of Sigma Phosphate Buffered Saline (PBS) tablets to 200 ml distilled H₂O, to give a 0.07 M Phosphate buffer at pH 7.4 with 0.96 M NaCl. Add 5.6 gm Sucrose (41 mM Sucrose) to the PBS solution. For long term stability the buffer is frozen. MSB is stable for about 30 days in refrigerator.

### Preparation of GnRH/KLH Conjugate:

KLH carrier protein is first subjected to maleimide activation for addition of sulfide binging groups thereto. 10 mg of the mollusk protein KLH is dissolved in the MSB buffer, and 2 mg of sulfo-SMCC is added (Pierce Chemical Co.) with gentle mixing to dissolve. The mixture is allowed to react for 1 hour at room temperature with periodic mixing. After completion of the reaction, the maleimide-activated protein is immediately purified by applying the reaction mixture to a desalting column (i.e., Sephadex G-25). The maleimide activated protein comes off on the void volume (first peak, fractions 4-6) as measured by absorbance at 280 nm. There is a drop in absorbance after fraction 6, and a rise in absorbance in fractions 7 or 8 as the excess sulfo-SMCC comes off. Excess cross-linker is removed in order to achieve good conjugation to the hapten. At this point the maleimide activated KLH may be frozen or freeze dried.

### GnRH Hapten Conjugation:

Six mg of the GnRH-Gly-Cys hapten (containing an free SH on one end) is dissolved in 1 ml of H₂O. The dissolved hapten is added to the activated KLH and allowed to react for 2 hours at room temperature and then overnight in the refrigerator. The KLH-hapten conjugate is immediately purified by applying the reaction mixture to a desalting column. (Sephadex G-25). The conjugate protein should come off on the void volume (first peak, fractions 4-6) as measure by absorbance at 280 nm. There should be a drop in absorbance after fraction 6 and rise in absorbance in fractions 7 or 8 as the excess hapten comes off. A small amount of excess hapten does not cause problem in a prime dose but could neutralize antibody in a boost dose if it is present in large excess.

### GnRH Vaccine Formulation:

Primary dose formulations of the vaccine are prepared by mixing equal portions (1:1 ratio) of GnRH-KLH conjugate (0.5 ml) with the prime dose adjuvant of Example 1 (1/150) (0.5 ml). The final vaccine dose should contain approximately 170 μg of killed bacteria per 0.5 ml dose. The GnRH conjugate must be added to the oil adjuvant (not oil to GnRH) in a drop wise manner while the oil is vortexed. This forms a milk like emulsion. The emulsion is stiffened by passing through a 22 gauge needle 3 times.

Boost dose formulations of the vaccine are prepared by mixing equal portions (1:1 ratio) of GnRH-KLH vaccine (0.5 ml) with the boost dose adjuvant of Example 1 (1/300) (0.5 ml). The final vaccine dose should contain approximately 85 μg of killed bacteria per 0.5 ml dose. Again, the GnRH conjugate must be added to the oil adjuvant (not oil to GnRH) in a drop wise manner while the oil is vortexed. This forms a milk like emulsion. The emulsion is stiffened by passing through a 22 gauge needle 3 times.

### Example 3

### GnRH-Blue Vaccine Preparation Using Non-Activated Blue Protein

### Preparation of Mollusk Stabilizing Buffer (MSB):

Dissolve 7 tablets of Phosphate Buffered Saline (PBS) (Sigma Chemical Co., P-4417) into 400 mL distilled water to yield a 30 nM phosphate buffer with 0.4 M NaCl and a pH of 7.4. Dissolve 5.6 g of sucrose (Sigma Chemical Co., S-9378) into the PBS solution to yield 41 mM sucrose. The formulation may be stored at a temperature in the range of 3−8° C. for 90 from the date of manufacture.

### Activation and Purification of Non-Activated Blue Protein:

For each 100 mL of vaccine volume required, add a 10 mg mass of Sulfo-SMCC to a glass culture tube and dissolve using 3 mL of distilled water (the resultant solution should be clear). Add 1 mL (MSB) to the Sulfo-SMCC solution in each tube (the resultant solution should remain clear). Add 80 mg of stock non-activated Blue protein to the tube while mixing. Incubate the mixture for approximately an hour at room temperature (20-25° C.) with periodic mixing to activate the Blue and Sulfo-SMCC solution. The total volume of the tube will be approximately 4.5 mL, dependent upon the stock concentration of the Blue protein.

Quality of the Blue and Sulfo-SMCC solution is checked by making a 1:100 dilution of the Blue and Sulfo-SMCC solution with MSB and mixing well (for example, 10 μL of solution mixed with 990 μL of MSB). Blank a spectrophotometer with a UV compatible cuvette (quartz or UV-safe plastic) containing MSB at a wavelength of 280 nm. Measure absorbance of 1 mL of the 1:100 dilution of the activated Blue and Sulfo-SMCC solution at the 280 nm wavelength with the blanked spectrophotometer. If the absorbance is 0.4±0.1 the process can proceed. If the absorbance is outside of 0.4±0.1 the activated Blue and Sulfo-SMCC solution must be disposed of and remade.

The activated Blue and Sulfo-SMCC solution may be purified by transferring each glass culture tube of the solution to a separate length of 3,500 MWCO dialysis tubing (Spectrum Laboratories, Inc., Spectra/Por 3 or equivalent) and individually dialyzing each tube of the solution against 2 L of lxPhosphate-Buffered Saline (PBS) for approximately 2 hours at room temperature with gentle stirring on a magnetic stir plate (2 L of 1×PBS may be prepared by dissolving 10 PBS tablets in 2 L of distilled water, the resulting solution will be 0.01 M phosphate buffer, 0.0027 M potassium chloride, and 0.137 M sodium chloride with a pH of 7.4). After 2 hours of dialysis, the solution in each dialysis tube is transferred to 2 L of 1×PBS made in a clean tube and dialyzed for approximately 2 hours at room temperature with gentle stirring on a magnetic stir plate. After completion of dialysis, each tube of activated and purified Blue and Sulfo-SMCC solution is transferred to individual 50 mL centrifuge tubes and QS to 15 mL with MSB.

Quality of the activated and purified Blue and Sulfo-SMCC solution is checked by making a 1:10 well mixed dilution of purified and activated Blue and Sulfo-SMCC solution with MSB (for example, 100 μL of Blue and Sulfo-SMCC solution mixed with 900 μL MSB). Blank a spectrophotometer with a UV compatible cuvette (quartz or UV-safe plastic) containing MSB at a wavelength of 280 nm. Measure absorbance of 1 mL of the 1:10 dilution of the purified and activated Blue and Sulfo-SMCC solution at the 280 nm wavelength with the blanked spectrophotometer. If the absorbance is 0.6±0.1 the process can proceed. If the absorbance is outside of 0.6±0.1 the purified and activate Blue and Sulfo-SMCC solution must be disposed of and remade.

### Conjugation of Blue Protein and GnRH:

For each 100 mL vaccine volume required, dissolve a 30 mg mass of GnRH-Gly-Cys-hapten with free sulfhydryl (—SH) at C-terminus (GL Biochem (Shanghai) Ltd., custom synthesis >90% pure by HPLC)(hereinafter “GnRH1”) to a glass culture tube and dissolve in 1 mL of distilled water.

A quality of the GnRH1 solution may be checked by mixing 100 μL of a solution containing 1 mg of Ellman's reagent per 1 ml 1×PBS with 10 μL of the GnRH1 solution in a clean glass culture tube. A bright yellow color indicates that free —SH are present and the GnRH1 solution is satisfactory for conjugation. If the solution is not bright yellow, the test should be repeated with fresh Ellman's solution. If the test fails again, the GnRH1 solution is not satisfactory for conjugation and should be disposed of as medical waste.

If the GnRH1 is satisfactory for conjugation, add 1 mL of the 30 mg/mL GnRH1 solution to each tube containing 15 mL of purified and activated Blue and Sulfo-SMCC solution, and mix gently. Observe for an immediate flocculation and cloudiness upon addition of the GnRH1 solution to each tube of the purified and activated Blue and Sulfo-SMCC solution. If flocculation does not occur, do not proceed any further with the conjugation and dispose of the solution as medical waste. If flocculation occurs, allow the conjugation reaction to continue at room temperature for approximately 2 hours with occasional mixing. After 2 hours, transfer the reaction to a temperature of 3°−8° C. overnight to complete conjugation. Following overnight conjugation, QS the Blue-GnRH1 conjugate in each tube to 50 mL with MSB.

### Preparation of the Primary Dose Adjuvant:

Prepare adjuvant stock solution and a mineral oil diluent as discussed in Example 1 above. Combine 10 mL of adjuvant stock solution with 40 mL mineral oil diluent in a 50 mL centrifuge tube and mix thoroughly. The resulting primary dose adjuvant will have a concentration of Mycobacterium avium at 0.332 mg/mL. When stored at 3-8° C., the solution will expire two years from the date of manufacture.

### Emulsification of Blue-GnRH Conjugate and the Primary Dose Adjuvant:

Transfer 50 mL of the primary dose adjuvant to a 200-400 mL capacity glass beaker and create a strong vortex (the vortex may be created with a propeller-style mixing blade of a stand mixer operating at approximately 600 RPM). Create a primary emulsion by adding the Blue-GnRH1 conjugate to the vortex in a dropwise manner while slowly increasing the mixing speed as the Blue-GnRH1 conjugate is added to maintain a strong vortex as the emulsion thickens and volume increases. Each 50 mL volume of the conjugate must be emulsified separately. This primary emulsion may be temporarily stored at 3-8° C. if not being processed in the Microfluidizer immediately.

Pool all the primary emulsions in one beaker (approximately 100 ml of the primary emulsion) and mix approximately 2 minutes on medium speed using a stand mixer. Slowly move the mixing blade up and down in the emulsion to make sure it is evenly mixed while not introducing air bubbles into the emulsion.

A secondary emulsion may be prepared using a microfluidizer. Prior to running the primary emulsion through the microfluidizer, run approximately 200 mL of 70% isopropanol through the system. Then flush the isopropanol from the line by running approximately 100 mL of MSB through the system twice. Transfer the primary emulsion to a 50 or 100 mL glass syringe. Seat the syringe containing the primary emulsion onto the microfluidizer and prime the microfluidizer by running the emulsion trough at 6000 PSI. Turn off the microfluidizer once primed.

Attach a disposable, rubber-free 60 mL syringe to the microfluidizer output and collect the secondary emulsion. Repeat as necessary until all the primary emulsion has been converted to the secondary emulsion.

The secondary emulsion is the final vaccine product. The final product can be loaded into syringes of appropriate dosages and stored at 3-8° C. for 6 months after manufacture.

### Example 4

### GnRH-Blue Vaccine Preparation Using Activated Blue Protein and GnRH-KLH Vaccine Preparation

Prepare Mollusk Stabilizing Buffer (MSB) as discussed above in Example 3.

For each 100 mL vaccine volume required, rehydrate 6×10 mg vials of lyophilized maleamide-activated Blue protein (Blue) or mcKLH (KLH) carrier protein with 1 mL of deionized water each (the carrier protein must be activated with at least 400 mol maleamide/mol). For each 100 mL vaccine required, dissolve 30 mg of GnRH in a culture tube using 1 mL of distilled water.

Quality of the GnRH solution may be checked by mixing 100 μL of a solution containing 1 mg of Ellman's reagent per 1 ml 1×PBS with 10 μL of the GnRH solution in a clean glass culture tube. A bright yellow color indicates that free —SH are present and the GnRH solution is satisfactory for conjugation. If the solution is not bright yellow, the test should be repeated with fresh Ellman's solution. If the test fails again, the GnRH solution is not satisfactory for conjugation and should be disposed of as medical waste.

Add 1 mL of the satisfactory GnRH solution to 5 mL of MSB in a culture tube and mix well. Add 1 mL of the GnRH-MSB solution to each of the 6 rehydrated Blue or mcKLH vials. If flocculation does not occur in the vials, do not proceed any further with the conjugation and dispose of the solution as medical waste. If flocculation occurs, allow conjugation reaction to continue at room temperature for approximately 2 hours with occasional mixing.

For each 100 mL batch, combine the 6 vials of Blue-GnRH or mcKLH-GnRH conjugate in a 50 mL centrifuge tube. Rinse each vial once with 2 mL of MSB and add the rinsate to the conjugate in the 50 ml centrifuge tube for a total volume of 24 mL. QS the tube to 50 mL with MSB and gently mix by inversion.

Prepare the primary adjuvant as discussed in Example 3 above and emulsify the conjugate in the adjuvant as discussed in Example 3 above to prepare the final vaccine product.

### Example 5

### Immunocontraception of Deer

The GnRH immunocontraceptive vaccine of Example 2 was used for the immunocontraception of deer as either a two shot or single shot vaccine.

### Two Shot Trial:

Deer were injected with a first, prime boost, followed by injection 1 year later with a second, boost injection of the GnRH/adjuvant vaccines of Example 1. The deer were injected with 1 ml of the vaccine composition. Titers of anti-GnRH antibodies and blood progesterone levels were monitored over a two year period immediately prior to and following treatment. The amount of the conjugate in each dose of the vaccine was 450 μg.

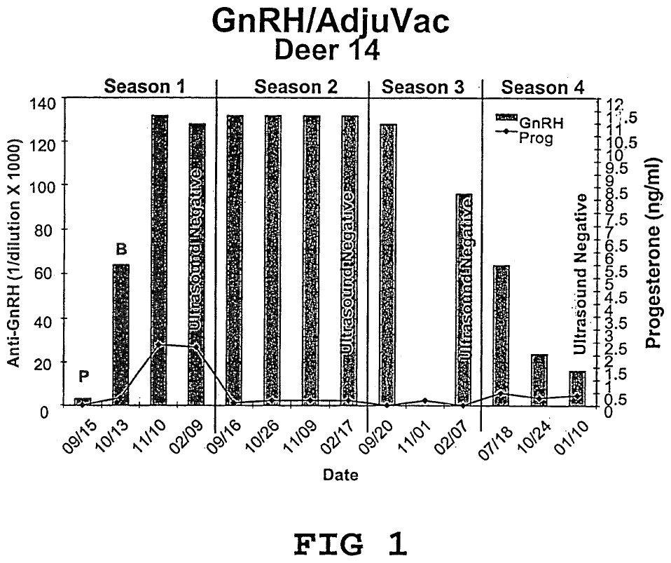

The results are shown in FIG. 1. Deer injected with a prime and boost vaccination of KLH-GnRH/Adjuvant in the breeding season of the first year of the trial have remained infertile through four consecutive breeding seasons (four years) without a second or third season boost vaccine. Anti-GnRH antibody titers remained at 128,000 into the third year, and dropped to 28,000 in the fourth year with deer remaining infertile (FIG. 1). The two shot paradigm effectively contracepted deer from 2 to 4 years. Two out of the 3 deer tested were still infertile after 4 years.

One Shot Trial with Female Deer:

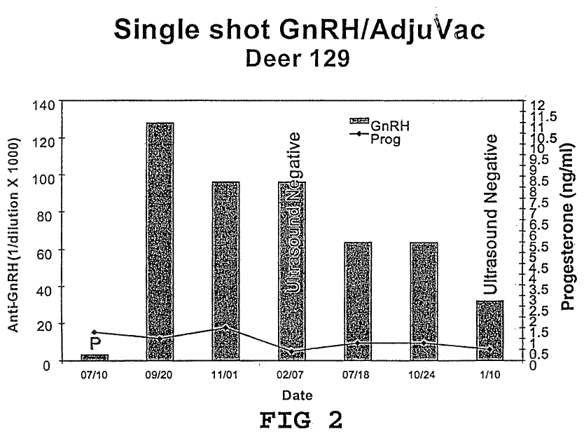

Following the success observed after two mating seasons of the two shot trial, a second trial was commenced to determine efficacy using only a single shot of the vaccine of Example 2. In July prior to the first mating season of this single shot trial, 5 Penn State deer were injected with 1 ml of the vaccine composition. The dose of the GnRH conjugate was increased over that used in the two shot trial; the concentration of the GnRH concentration was 850 μg/ml. They were exposed to the bucks on November of that year. All five remained infertile for that year. FIG. 2 represents a typical antibody response for the single shot deer. In the 2nd year 3 out of the 5 remained infertile, while two of the deer had a single fawn indicating a partial protection.

Single Shot Trial with Male Deer:

One season after the single shot trial was initiated with female deer, male deer were subjected to a single shot trial. In July prior to the first mating season of this trial, 5 male deer were injected with a single shot GnRH using the same amounts and concentrations of vaccine as in the single shot trial with the females (1 ml dose containing 850 μg of conjugate). In the November bleed the testosterone levels of all 5 deer were down to the level of sexually immature deer, and their antlers had prematurely dropped off. Therefore the single shot regimen was effective in shutting the sexual activity of all male and female deer for at least one year (Table 1).

TABLE 1
White Tail Bucks
Single Shot

| | | Testosterone | Testis | |
|---|---|---|---|---|
| | Anti-GnRH | (mg/dl) | (μm) | Antlers |
| Control | 0 ± 0 | 477 ± 172 | 73 ± 43 | hardened |
| One Shot | 48K ± 23K | 4 ± 6 | 44 ± 28 | velvet or shed |
| regimen | | | | |

### Example 6

### Immunocontraception of Pigs

The GnRH immunocontraceptive vaccine of Example 2 was used for the immunocontraception of pigs as a single shot vaccine.

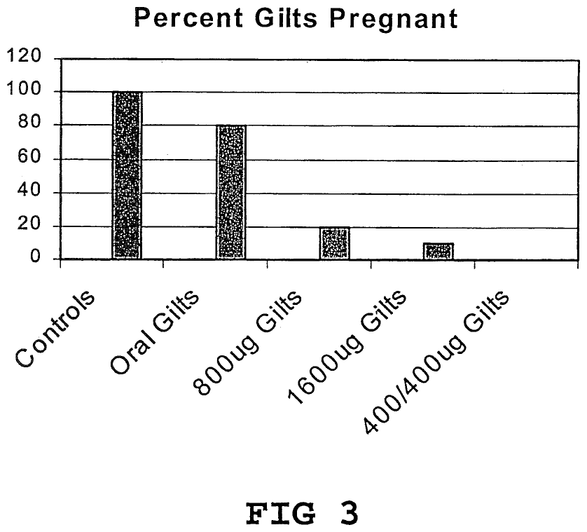

The GnRH/adjuvant vaccine was tested in fifty 5 month old gilts. Pigs of Group 1 (n=10) were sham injected with the adjuvant only, while pigs of Group 2 (n=10) were given 2 oral doses of GnRH on mixed nut shells, Group 3 (n=10) were given a single dose containing 800 μg of GnRH conjugate, Group 4 (n=10) were given a single dose of 1600 μg of GnRH conjugate, and Group 5 (n=10) were given 2 doses of 400 μg of GnRH conjugate 30 days apart. At eight months of age or 3 months after the contraceptive vaccine was given the gilts were checked for heat by teasing with a boar. The results which are dose related are shown in FIG. 3. The 2 dose trial was the most effective giving a 100% contraceptive effect. However, the high single dose gave 90% contraceptive effect response.

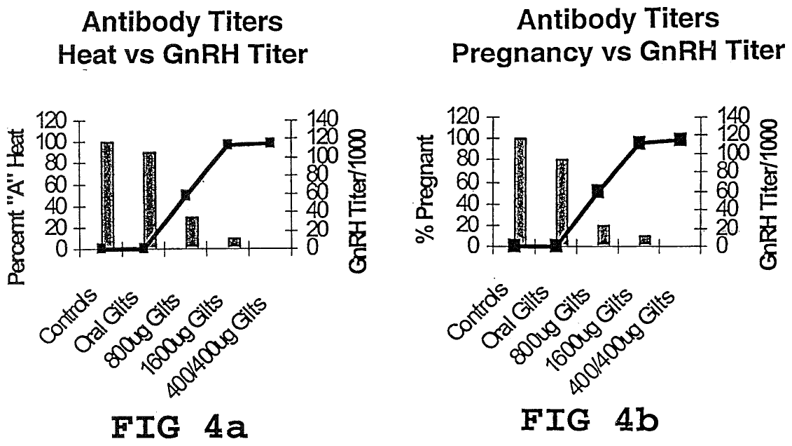

As seen in FIG. 4 the heat cycles and pregnancy observed in the 8 months old female gilt decreased as the GnRH antibody titer increased.

It is understood that the foregoing detailed description is given merely by way of illustration and that modifications and variations may be made therein without departing from the spirit and scope of the present subject matter.

We claim:

1. A method for immunocontraception of an animal which comprises administering an immunocontraceptive vaccine composition to said animal which comprises:
a. an immunogen comprising GnRH or a GnRH immunogenic analog conjugated to a carrier protein, and
b. an adjuvant comprising mineral oil and killed cells of Mycobacterium avium subspecies avium or Mycobacterium avium complex, the concentration of said killed cells of Mycobacterium avium being in an amount effective for inducing immunocontraception of said animal in a single dose.

2. The method of claim 1, wherein said vaccine composition is administered in a single dose, without a second or boost dose, for a period of at least one year.

3. The method of claim 1, wherein a second or boost dose of said vaccine composition is administered to said animal within one year of administration of a first dose of said vaccine composition to said animal.

4. The method of claim 1 wherein the concentration of said killed cells of Mycobacterium avium subspecies avium or Mycobacterium avium complex in said vaccine composition is greater than or equal to about 50 μg per ml and less than or equal to about 400 μg per ml, measured as the dry weight of said killed cells per ml of said vaccine composition.

5. The method of claim 4 wherein the amount of said killed cells of Mycobacterium avium subspecies avium or Mycobacterium avium complex in said vaccine composition is less than or equal to about 400 μg, measured as the dry weight of said killed cells.

6. The method of claim 5 wherein the amount of said killed cells of Mycobacterium avium subspecies avium or Mycobacterium avium complex in said vaccine composition is less than or equal to about 200 μg, measured as the dry weight of said killed cells.

7. The method of claim 1 wherein said animal is selected from the group consisting of porcine, bovine, equine, feline, canine, primates, Rodentia, Cervidae, and Pachydermata.

8. The method of claim 1 wherein said animal is selected from the group consisting of domestic dogs, domestic cats, pigs, cattle, deer, horses, zoo animals, elephants, rodents, and reptiles.

9. The method of claim 1 wherein said adjuvant further comprises a surfactant.

10. The method of claim 1 wherein said surfactant is selected from the group consisting of mannide monooleate, isomannide monooleate, aluminum monostearate, and combinations thereof.

11. The method of claim 1 wherein said surfactant is mannide monooleate.

12. The method of claim 1 wherein said immunogen further comprises an antibiotic or a preservative.

13. The method of claim 1 wherein said immunogen comprises GnRH or a GnRH immunogenic analog conjugated to a KLH carrier protein or a Blue carrier protein, and said GnRH or GnRH immunogenic analog is conjugated to said KLH carrier protein or said Blue carrier protein through a C-terminal end of said GnRH or GnRH immunogenic analog.

14. The method of claim 13 wherein said immunogen comprises GnRH or a GnRH immunogenic analog conjugated to a Blue carrier protein, and said GnRH or GnRH immunogenic analog is conjugated to said Blue carrier protein through a C-terminal end of said GnRH or GnRH immunogenic analog.

15. The method of claim 13 wherein said vaccine composition further comprises physiologically buffered saline, and further wherein the salt concentration of said vaccine composition is greater than or equal to about 0.7 M and less than or equal to about 1.0 M, and the pH of said vaccine composition is between about 7.0 and 8.0.

16. The method of claim 1 wherein said vaccine composition is administered by intermuscular injection.

17. The method of claim 1 wherein the amount of said killed cells of Mycobacterium avium in said vaccine composition is not sufficient to elicit a substantial T cell-mediated delayed hypersensitivity response to M. avium by said animal if said adjuvant was administered alone, without said immunogen.

18. The method of claim 1 wherein said animal is selected from the group consisting of deer, domestic cats, squirrels, horses, and elk.
